(19) United States
(12) Patent Application Publication
(10) Pub. No.: US 2003/0022333 A1
(43) Pub. Date: Jan. 30, 2003

(54) Long-term shelf preservation by vitrification

(76) Inventor: Victor Bronshtein, Rochester, NY (US)

Correspondence Address: Barbara E. Johnson, Webb Ziesenheim Logsdon Orkin & Hanson, 700 Koppers Building, 436 Seventh Avenue, Pittsburgh, PA, 15219, US

(21) Appl. No.: 10/174,007

(22) Filed: Jun. 18, 2002

Related U.S. Application Data
(63) Continuation of application No. 09/734,970, filed on Dec. 12, 2000, now abandoned.
(63) Continuation of application No. 08/785,472, filed on Jan. 17, 1997, now abandoned.
(60) Provisional application No. 60/018,573, filed on May 29, 1996.

(51) Int. Cl. C12N 9/96; A01N 1/00; C12N 1/04; C12N 5/00; C12N 5/02

(52) U.S. Cl. 435/188; 435/374; 435/420; 435/260; 435/1.1

(57) ABSTRACT

A method of shelf preserving biologically active specimens by vitrifying them, i.e., dehydrating them in such a way as to achieve a true glass state at storage temperature by subsequent cooling. The method is founded upon the recognition that to store samples in a true glass state the dehydration temperature of the material to be dehydrated must be higher than the suggested storage temperature. Because the vitrification temperature quickly decreases with increasing water content (for example, pure water vitrifies at T_{g}=−145° C., whereas 80 percent by weight sucrose solution vitrifies at T_{g}=−40° C. and anhydrous sucrose vitrifies at T_{g}=60° C.) the sample needs to be strongly dehydrated to increase the T_{g} above the temperature of storage (Tₛ). As determined by the inventor, the dehydration temperature should be selected as higher than the suggested storage temperature, and the glass state is subsequently achieved by cooling after dehydration.

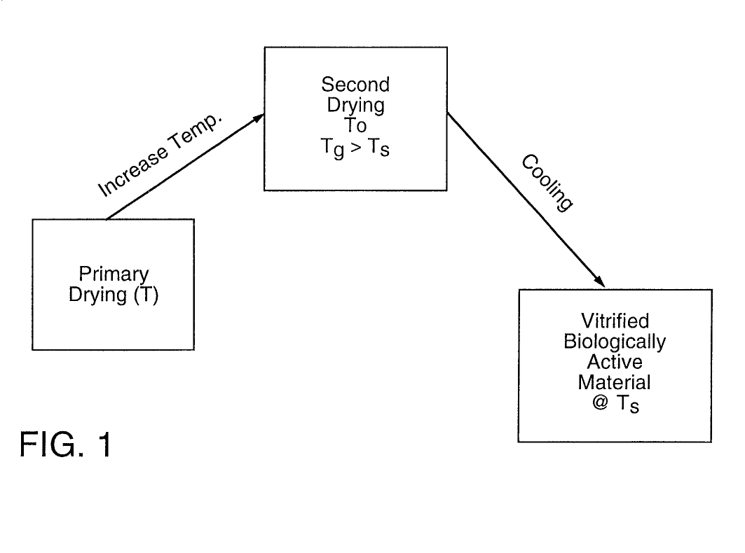

### CROSS REFERENCE TO RELATED APPLICATIONS

This application is a continuation of U.S. Pat. application Ser. Nos. 09/734,970, filed Dec. 12, 2000; 08/785,472, filed Jan. 17, 1997; all of which claim priority to U.S. Provisional Application Serial No. 60/018,573, filed May 29, 1996.

### FIELD OF THE INVENTION

The invention relates to methods for preserving solutions and emulsions of suspended or dispersed molecules, especially biologically active molecules, and also cells and tissues, using improved vitrification techniques to achieve the true glass state for maximized storage stability.

### BACKGROUND OF THE INVENTION

The long-term storage of biologically active materials and cells and multicellular tissues is becoming more and more necessary for both commercial and research purposes, yet such materials may be the most difficult to store of any materials known. Ironically, the same properties which make biologically active agents and life forms valuable are the properties which make them so difficult to preserve. Certainly very few such materials are sufficiently stable to allow them to be isolated, purified and stored in room temperature solution for anything more than a very short period of time.

Both commercially and practically, shelf storage of dehydrated biologically active materials carries with it enormous benefits. Successfully dehydrated reagents, materials and cells have reduced weight and require reduced space for storage notwithstanding their increased shelf life. Room temperature storage of dried materials is moreover cost effective when compared to low temperature storage options and their concomitant costs. The biologically active materials addressed herein include, without limitation, biologically active macromolecules (enzymes, serums, vaccines), viruses and pesticides, drug delivery systems and liposomes, and cell suspensions such as sperm, erythrocytes and other blood cells, stem cells and multicellular tissues such as skin, heart valves and so on.

As the benefits of shelf preservation of biological specimens has become more appreciated, researchers have endeavored to harness “vitrification” technology in the biological world. The technology of “vitrifying,” or achieving the “glass” state for any given material, has thus been anticipated to emerge as a premier preservation technique for the future, although prior art vitrification techniques have been plagued with unexpected problems. As the developments underlying the invention will illustrate, although Applicant does not intend to be bound by this theory, in retrospect it would appear that fear of sample damage has inhibited previous investigators from considering appropriate temperatures for dehydration in order truly to achieve the glass state of any given material at ambient temperature. As a result, previous attempts at vitrification have generally yielded inferior products, with excessive water content or having properties inconsistent with a true glass state. These products generally exhibit limited storage stability at room or higher temperature.

An important misconception has inhered in the belief that vitrification can be achieved by drying alone. References are numerous in which substances are purported to have achieved a true glass state by drying, yet the disclosed techniques do not actually result in a glass state's forming. The true statement is that because drying is a process limited by diffusion of water molecules, the glass state at constant hydrostatic pressure can be achieved only by cooling (although prior to the present invention this was not appreciated). In this context, it is important to note issued p atents in which this misconception is misleadingly embodied. Wettlaufer and Leopold, U.S. Pat. No. 5,290,765, patented a method of protecting biological materials from destructive reactions in the dry state. They suggest to protect the biological suspension during drying and subsequent storage by combining the suspensions with sufficient quantities of one or more vitrifying solutes and recommended a 3/1 weight percent sucrose/raffinose mixture. The materials are taught as intended to be dried until drying is sufficient, but this is misleading and an erroneous teaching. At best, these materials achieve a very viscous liquid state which resembles a rubbery state, but no glass state ever emerges.

Franks et al., in U.S. Pat. No. 5,098,893, likewise teaches that all that is necessary to achieve the glass state at ambient temperature is evaporation at ambient temperature and that any optional temperature increase should be imposed only to increase evaporation rate. For this reason, even though Franks et al. believe that the samples described in their examples achieve the glass state, in actuality they do not.

The misconception explained above has occurred for several reasons. First, some individuals have used the terms “glass,” “glassy” and/or “vitrified” in a vague and hence misleading way. Second, it is admittedly difficult to measure reliably the glass transition temperature of dry mixtures containing polymers or biopolymers. The change in specific heat in such mixtures is very small and occurs over a broad temperature range that makes reliable differential scanning calorimetry (DSC) measurements of T_{g} difficult. When the measurement is omitted, certain individuals assume that a glass state has been achieved when it has not. Third, sometimes more water remains in a supposedly vitrified material than would be consistent with a true glass state, but in many cases measurement of this water for a variety of reasons gives an erroneous result. All of these reasons, and probably others, tend to fuel the wishful thinking that a glass state has been achieved when it in fact has not. Because the diffusion coefficient of water quickly increases with increasing temperature above the glass transition temperature, with prior art preservation methods the safe storage time is limited if samples are stored above the glass transition temperature.

A need thus remains for a preservation protocol which effects true vitrification of biologically active materials including peptides, proteins, other molecules and macromolecules and also cells, to provide unlimited storage time.

SUMMARY OF THE INVENTION

In order to meet this need, the present invention is a method of shelf preserving biologically active specimens by vitrifying them, i.e., dehydrating them in such a way as to achieve a true glass state. The method is founded upon the recognition that to store samples in a true glass state the dehydration temperature of the material to be dehydrated must be higher than the suggested storage temperature. Because the vitrification temperature quickly decreases with increasing water content (for example, pure water vitrifies at T_{g}=−145° C., whereas 80 percent by weight sucrose solution vitrifies at T_{g}=−40° C. and anhydrous sucrose vitrifies at T_{g}=60° C.) the sample needs to be strongly dehydrated to increase the T_{g} above the temperature of storage (Tₛ). As determined by the inventor, the dehydration temperature should be higher than the suggested storage temperature and the glass state should be subsequently achieved by cooling after dehydration. For example, implementing this directive in some cases requires only drying at room temperatures followed by cooling to a lower-than-room-temperature storage temperature; in other instances the present method requires careful heating of the substance to be vitrified to a temperature above room temperature, followed by dehydration and subsequent cooling to room temperature.

### DETAILED DESCRIPTION OF THE INVENTION

The invention described herein overcomes the deficiencies of the prior art and allows preservation and storage of specimens in the actual glass state without loss of biological activity during storage. Biological specimens which can be vitrified to a glass state include, without limitation, proteins, enzymes, serums, vaccines, viruses, liposomes, cells and in certain instances certain multicellular specimens. The shelf storage time in the glass state is practically unlimited and there is no need to perform accelerated aging to estimate the safe storage time. The key to genuine vitrification is to conduct the dehydration at a temperature higher than the suggested storage temperature (Tₛ) to achieve the glass transition temperature (T_{g}, T_{g}>Tₛ) followed by cooling of the sample to the suggested storage temperature, Tₛ. As an example, implementing this protocol in some cases requires only dehydration at room temperature followed by cooling to a lower-than-room-temperature storage temperature; in other instances the present method requires careful dehydration of the substance to be vitrified to a temperature above room temperature, followed by cooling to room temperature.

This invention may be used to provide unlimited shelf storage of biological specimens by vitrification at intermediate low (refrigeration) temperatures (more than −50° C.) and/or ambient or higher temperatures. It is then possible to reverse the vitrification process to the preserved sample's initial physiological activity. The method may be applied for stabilization of pharmaceutical and food products as well.

In its broadest sense, vitrification refers to the transformation of a liquid into an amorphous solid. While liquid-to-glass transition may not yet be completely understood, it is well established that liquid-to-glass transition is characterized by a simultaneous decrease in entropy, sharp decreases in heat capacity and expansion coefficient, and large increases in viscosity. Several microscopic models have been proposed to explain liquid-to-glass transition, including free volume theory, percolation theory, mode coupling theories and others. Theories are unimportant, however, as long as the practice of the invention reliable experimental methods for establishing T_{g} are used. The recommended method is the temperature stimulated depolarization current method known in the art.

To improve quality and prolong unlimited shelf life at storage temperatures, the samples should be dehydrated so that T_{g} actually becomes higher than Tₛ. Depending on the suggested Tₛ, value, different dehydration methods may be applied. For example, freezing may allow storage at a temperature less than T_{g}, which is the vitrification temperature of the maximum freeze dehydrated sample (or solution). Appropriate dehydration according to the invention may allow storage at ambient temperatures. However, because dehydration of the glassy materials is practically impossible, the only way to achieve T_{g}>Tₛ at constant hydrostatic pressure is to dehydrate the samples at a temperature that is higher than the glass transition temperature. This has to be done despite risk of heat degradation of the specimen.

Dehydration of biological specimens at elevated temperatures may be very damaging if the temperatures used are higher than the applicable protein denaturation temperature. To protect the samples from the damage associated with elevation of temperature, the dehydration process should be performed in steps. The first step of the dehydration (air or vacuum) should be performed at such low temperatures that the sample can be dehydrated without loss of its activity. If the first step requires dehydration at sub-zero temperatures one may apply freeze-drying techniques. After the first drying step, the dehydration may be continued by drying at higher temperatures. Each step will allow simultaneous increases in the extent of dehydration and temperature of drying. For example, in the case of enzyme preservation it was shown that after drying at room temperature the drying temperature may be increased to at least 50° C. without loss of enzymatic activity. The extent of dehydration obtained after drying at 50° C. will allow a further increase in the drying temperature, without loss of activity. For any given specimen to be preserved, the identity of the specimen will determine the maximum temperature it can withstand during the preservation process, i.e., denaturation temperature, etc. It should be noted, however, that various protectants and cryoprotectants confer protection to materials to be dried during the drying process, i.e., sugars, polyols and polymeric cryoprotectants.

It should also be noted that, according to the invention, all methods of successful freeze-drying and drying of biological specimens reported so far can be optimized by the additional vitrification according to this invention. The vitrified samples can then be stored on a shelf for an unlimited time. The only negative effect of actual vitrification may be increasing the time of dissolution in water or rehydrating solution, which in itself may cause certain damage to some specimens in some cases. It is possible to ameliorate this unwanted effect by judicious heating of the rehydration water prior to its application to the vitrified specimen. Heating is judicious when it is controlled within limits which minimize sample damage.

Although the invention has been described in terms of particular materials and methods above, the invention is only to be limited insofar as is set forth in the accompanying claims.

I claim:

12. A method of shelf preservation of biological specimens by true vitrification, comprising treating a sample including a biologically active material by:

1) drying the sample in a first primary drying step; and

2) continuing to dry said sample in a second drying step, with the drying temperature of said second drying step being higher than both the temperature of said first step and the storage temperature (Tₛ), with said second drying step continuing for a period of time sufficient to increase the glass transition temperature (T_{g}) of said sample to a point above said storage temperature (Tₛ) of said sample; followed by

3) cooling said sample to said storage temperature; wherein said drying and cooling steps yield a vitrified biologically active material.

13. The method according to claim 12, wherein said biologically active material is selected from the group consisting of enzymes, peptides, proteins, biological molecules, biological macromolecules and cells.

14. The method according to claim 12, wherein said biologically active material is selected from the group consisting of proteins, enzymes, serums, vaccines, viruses, liposomes, cells and multicellular specimens.

15. The method according to claim 12, wherein said biologically active material is combined with a protectant selected from the group consisting of sugars, polyols and polymers and further which is water soluble or water swellable.

16. The method according to claim 12, wherein said storage temperature exceeds about 20° C.

17. The method according to claim 12, wherein after a period of storage said sample is rehydrated.

18. The method according to claim 17, wherein said sample rehydrated with water having a temperature greater than the storage temperature of the sample.

19. The method according to claim 18, wherein said sample is stored at a temperature exceeding about 20° C.

20. The method according to claim 19, wherein said sample is stored at a temperature exceeding about 30° C.

21. The method according to claim 20, wherein said sample is stored at a temperature exceeding about 40° C.
